(19) United States
(12) Patent Application Publication
(10) Pub. No.: US 2023/0061465 A1
(43) Pub. Date: Mar. 2, 2023

(54) MEDICATION MONITORING AND ALERT SYSTEM

(71) Applicant: Hi.Q, Inc., Plano, TX (US)

(72) Inventor: Tom Schollmeyer, Plano, TX (US)

(21) Appl. No.: 17/892,417

(22) Filed: Aug. 22, 2022

Related U.S. Application Data
(60) Provisional application No. 63/236,995, filed on Aug. 25, 2021.

(51) Int. Cl. G16H 40/67; G16H 20/10; G08B 21/24

(52) U.S. Cl. CPC G16H 40/67; G16H 20/10; G08B 21/24

(57) ABSTRACT

An intelligent medication device includes a wireless sensing module that can sense medication events comprising medication consumption events and medication dispensing events in connection with a medication organizer. For each event, the wireless sensing module to verify the event using a medication schedule of a user of the medication organizer. When any errors are detected, the wireless sensing module can transmit an alert to one or more computing devices to indicate the error.

### CROSS-REFERENCE TO RELATED APPLICATION

This application claims the benefit of priority to U.S. Provisional Application No. 63/236,995, filed on Aug. 25, 2021; which is hereby incorporated by reference in its entirety.

### BACKGROUND

At home safety and monitoring devices typically utilize a combination of imaging, audio detection, and user interaction methods in order to, for example, provide assistance, alerts, entertainment, and/or security to users. However, the use of such active monitoring devices represents a tradeoff between smart home benefits and in-home privacy. Wireless sensing and artificial intelligence technology have opened the door to new possibilities in more passive but effective home monitoring and security with limited impact on privacy.

### BRIEF DESCRIPTION OF THE DRAWINGS

The disclosure herein is illustrated by way of example, and not by way of limitation, in the figures of the accompanying drawings in which like reference numerals refer to similar elements, and in which:

FIG. 1 illustrates an intelligent medication device implementing wireless signal monitoring and alerting, in accordance with examples described herein;

FIG. 2 is a block diagram illustrating a network-based computing system implementing a remote medication monitoring service, according to examples described herein;

FIG. 3 is a flow chart describing a method of wireless signal monitoring and verification of medication events, according to various examples;

FIG. 4 illustrates a computing device for communicating with the intelligent medication device and/or computing system, according to examples described herein; and

FIG. 5 is a block diagram that illustrates a computer system upon which examples described herein may be implemented.

### DETAILED DESCRIPTION

An intelligent medication device is described herein that includes a medication organizer comprising a plurality of compartments. Each of the plurality of compartments can hold a corresponding set of medication delivery means—such as pills, liquid capsules, injection devices, etc.—at any given time. The intelligent medication device can further include a wireless sensing module within wireless range of the medication organizer (e.g., coupled to its base). The wireless sensing module can include one or more wireless signal sensors and a network communication interface to communicate, over one or more networks, with one or more computing devices of a user of the medication organizer or one or more associates of the user (e.g., an emergency contact).

In various implementations, the wireless sensing module can determine a medication schedule for the user of the medication organizer. The medication schedule can correspond to the medications and dosages of the user for each day and/or each partial day of any given week. In certain examples, the wireless sensing module can generate, using a wireless signal generator, a wireless signal that penetrates each compartment of the plurality of compartments of the medication organizer. In variations, the wireless sensing module can utilize existing wireless signals (e.g., Wi-Fi signals) from various wireless devices, such as computers, routers, smart home devices, mobile device, and the like. The module may monitor, using the one or more wireless signal sensors, the corresponding set of medication delivery means in each of the plurality of compartments for medication consumption events by the user. In doing so, the sensing module can analyze reflected wireless signals from each of the plurality of compartments based on the wireless signal generated by the wireless signal generator, and based on the medication schedule for the user, verify each of the medication consumption events to determine whether an error or anomaly has occurred.

The intelligent medication device can further transmit one or more alerts indicating the error or anomaly to the computing device of the user or an associate of the user in response to determining that the error or anomaly has occurred. In further implementations, the intelligent medication device can further include a dispenser that selectively dispenses medication delivery means into each compartment of the medication organizer periodically in accordance with the user's medication schedule. According to examples provided herein, the wireless sensing module can further monitor the plurality of compartments of the medication organizer for dispensing events, and verify, using the medication schedule of the user, an accuracy of each detected dispensing event. For example, when a particular dispensing event comprises an inaccuracy, the sensing module can transmit an alert to the computing device of the user and/or an associate of the user to indicate the inaccuracy.

As described herein, each medication delivery means (e.g., comprising pills, liquid gels, injectors, disposable aerosols, etc.) can correspond to a predetermined wireless sensor signature which can indicate a unique density, size, shape, and/or property of the delivery means. In various examples, the wireless sensing module can verify each of the medication consumption events and/or medication dispensing events by comparing the sensor signatures of the medication delivery means in each of the plurality of compartments with the medication schedule of the user at any given time. For medication consumption events, the wireless sensing device can verify the identity of the user, verify that a correct compartment was opened, and verify whether the correct medications were consumed. In one example, the wireless sensing module can remain idle or in a low-power state until detecting a medication event trigger. Upon detecting an event trigger, the wireless sensing module can perform the verification processes accordingly

In variations, a computing system can implement a remote medication monitoring and verification service. The computing system can include a database storing user profiles of users of the remote medication monitoring and verification service. Each user profile can indicate a medication schedule of a respective user of the remote medication monitoring and verification service. The computing system can include a network communication interface to communicate, over one or more networks, with wireless sensing modules of intelligent medication devices of the users of the remote medication monitoring and verification service, and computing devices of the users and/or associates of the users (e.g., caretakers, family members, health care service providers, etc.

The computing system can receive event data indicating a medication event sensed by the wireless sensing module of an intelligent medication device of the respective user. In various examples, the event data can indicate one of (i) a medication consumption event by a respective user, or (ii) a medication dispensing event corresponding to medication being dispensed into a medication organizer of the respective user. Upon detecting the event, the computing system can analyze the event data using the medication schedule of the respective user to verify the medication event. Accordingly, when an error has occurred associated with the medication event, such as the respective user forgetting to take a particular medication or the dispenser dispensing one or more wrong medications into the medication organizer, the computing system can transmit an alert to a computing device associated with the respective user. The alert can indicate the error and even specify the nature of the error (e.g., a wrongly consumed pill or a wrongly dispensed medication into a compartment of the medication organizer).

In certain implementations, the event data can comprise raw sensor data from the wireless sensing module of the respective user. The raw sensor data can comprise reflected wireless signals detected by the wireless sensing module, which can indicate sensor signatures of the medications residing in each compartment of the respective user's medication organizer. As provided herein, based on the medication schedule of the respective user, the computing system can remotely analyze the raw sensor data to determine whether each particular compartment of the medication organizer has an accurate set of medication delivery means given the day of the week and/or time of day. In an example, an error can correspond to an inaccurate set of medication delivery means in the particular compartment at any given time.

In various examples, each medication delivery means can correspond to a sensor signature, and the computing system can remotely verify each detected medication event by comparing the sensor signatures of each medication delivery means in each compartment of the medication organizer with the medication schedule of the user. In further examples, the computing system can communicate with one or more health care providers of the respective user in order to, for example, determine the medication schedule of the respective user and/or any updates to the user's medication schedule. Upon receiving a medication update, the computing system can update the medication schedule in the user profile of the respective user accordingly. Thereafter, the computing system utilizes the updated medication schedule for the verification and alerting techniques described herein.

Examples described herein achieve a technical effect of utilizing wireless sensing technology to detect and verify medication events using a medication schedule of a user. In certain examples, the wireless sensing, verification, and/or alerting capabilities may be performed by a wireless sensing module on each intelligent medication device of each user. In variations, the wireless sensing module of each medication organizer can detect when a medication event occurs, detect and analyze wireless signals reflected from the medication organizer of the user, and transmit raw medication event data to a remote computing system, which then performs the verification and/or alerting tasks described herein.

As used herein, a computing device refers to devices corresponding to desktop computers, cellular devices or smartphones, personal digital assistants (PDAs), laptop computers, virtual reality (VR) or augmented reality (AR) headsets, tablet devices, television (IP Television), etc., that can provide network connectivity and processing resources for communicating with the system over a network. A computing device can also correspond to custom hardware, in-vehicle devices, or on-board computers, etc. The computing device can also operate a designated application configured to communicate with the network service.

One or more examples described herein provide that methods, techniques, and actions performed by a computing device are performed programmatically, or as a computer-implemented method. Programmatically, as used herein, means through the use of code or computer-executable instructions, and includes the implementation of machine learning and/or artificial intelligence to execute the processes described throughout the present disclosure. These instructions can be stored in one or more memory resources of the computing device. A programmatically performed step may or may not be automatic.

One or more examples described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines.

Some examples described herein can generally require the use of computing devices, including processing and memory resources. For example, one or more examples described herein may be implemented, in whole or in part, on computing devices such as servers, desktop computers, cellular or smartphones, personal digital assistants (e.g., PDAs), laptop computers, VR or AR devices, printers, digital picture frames, network equipment (e.g., routers) and tablet devices. Memory, processing, and network resources may all be used in connection with the establishment, use, or performance of any example described herein (including with the performance of any method or with the implementation of any system).

Furthermore, one or more examples described herein may be implemented through the use of instructions that are executable by one or more processors. These instructions may be carried on a computer-readable medium. Machines shown or described with figures below provide examples of processing resources and computer-readable mediums on which instructions for implementing examples disclosed herein can be carried and/or executed. In particular, the numerous machines shown with examples of the invention include processors and various forms of memory for holding data and instructions. Examples of computer-readable mediums include permanent memory storage devices, such as hard drives on personal computers or servers. Other examples of computer storage mediums include portable storage units, such as CD or DVD units, flash memory (such as carried on smartphones, multifunctional devices or tablets), and magnetic memory. Computers, terminals, network enabled devices (e.g., mobile devices, such as cell phones) are all examples of machines and devices that utilize processors, memory, and instructions stored on computer-readable mediums. Additionally, examples may be implemented in the form of computer-programs, or a computer usable carrier medium capable of carrying such a program.

Intelligent Medication Device

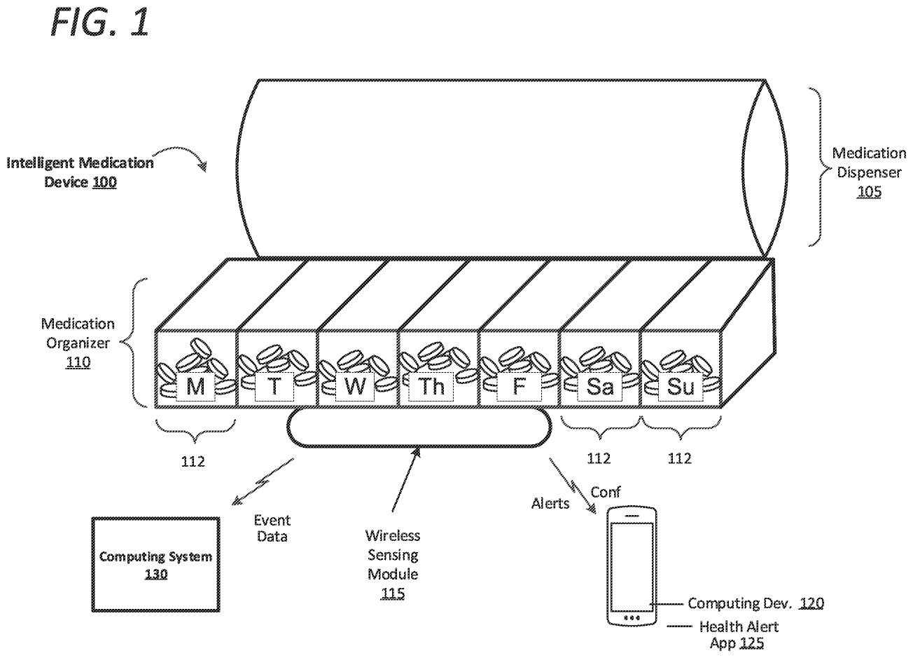

FIG. 1 illustrates an intelligent medication device 100 implementing wireless monitoring and alerting methods, in accordance with examples described herein. In various implementations, the intelligent medication device 100 can comprise a pill box, or medication organizer 110, that includes multiple compartments 112 each including a number of medication delivery means, such as pills, liquid gels, injection devices, etc. At any given time, the medication organizer 110 should have a correct amount of medication delivery means in each compartment 112. In certain examples, the intelligent medication device 100 can further include a medication dispenser 105. The medication dispenser 105 can include some or all of a user's medications, and can dispense the correct amount and type of medication delivery means into each compartment 112, either automatically (e.g., once a week or daily) or upon receiving a manual input.

It is contemplated that automatic dispensing of the medication delivery means into the medication organizer 110 may not be reliable. For example, at any given time, the dispenser 105 may not have a sufficient amount of a certain medication when a dispensing event occurs, which can result in an incorrect amount and/or type of medication delivery means in a particular compartment 112. Furthermore, the user may not consume the correct amount and/or type of medication on any given consumption event, which cannot be monitored through traditional means.

According to examples described herein, the intelligent medication device 100 can include a wireless sensing module 115 that can utilize wireless artificial intelligence technology to detect motion and track objects within each compartment 112 of the medication organizer 110. In certain examples, the wireless sensing module 115 can function as a verification tool to ensure that the correct type and amount of medication is in each compartment 112 of the medication organizer 110 at any given time. In various examples, the wireless sensing module 115 can obtain a medication schedule of the user, which can indicate the user's required intake of medication delivery means for each day of the week, and/or for specified times on specified days.

The wireless sensing module 115 may be powered by one or more batteries and/or may include a charging port to be powered by a wall outlet. Additionally or alternatively, the wireless sensing module 115 can include a wireless charging means, such as an induction coil for wireless charging.

In various implementations, the wireless sensing module 115 can include a wireless signal sensor that can detect reflected wireless signals (e.g., Wi-Fi, Bluetooth, etc.) and a processor to analyze the reflected wireless signals to identify the contents of each compartment 112 of the medication organizer 110. In one example, the wireless sensing module 115 can do so by utilizing the wireless signals from external devices, such as cellular phones, Wi-Fi routers, smart home devices, and the like. In variations, the wireless sensing module 115 can include a wireless signal generator that can output a wireless signal (e.g., Wi-Fi, Bluetooth, etc.) and detect and analyze the reflections of the outputted signals to determine the contents of each compartment 112 of the medication organizer 110.

As shown in FIG. 1, the wireless sensing module 115 is coupled to the medication organizer 110. However, the wireless sensing capabilities of the sensing module 115 are sufficiently granular such that the sensing module 115 may be located within a certain proximity of the medication organizer 110 (e.g., within the same room), and still effectively verify and track the medications within each compartment of the medication organizer 110. Accordingly, the wireless sensing module 115 need not be physically coupled to the medication organizer 110.

In certain examples, the wireless sensing module 115 can execute signal processing logic to process the reflected wireless signals in accordance with sensor signatures for each medication delivery means. Using the medication schedule of the user, the wireless sensing module 115 can, at any given time, process each sensor signature of each medication within each compartment 112 to verify whether the correct type and amount of medication delivery means is in each compartment 112. Accordingly, the wireless sensing module 115 can not only detect an amount of medication in each compartment 112, but can also determine each type of medication due to the unique size, shape, density, and/or property of each medication.

As such, the wireless sensing module 115 can be programmed to recognize the unique sensor signatures of any number of medications, each comprising a set of unique attributes corresponding to size, shape, etc. In certain examples, the wireless sensing module 115 can be triggered to verify a specific compartment 112 or the entire medication organizer 110 based on a triggering event. For example, the wireless sensing module 115 can detect when the user interacts with the medication organizer 110 (e.g., opens a particular compartment) and consumes at least a portion of the medications within a compartment 112. In variations, the intelligent medication device 100 can include one or more IMU sensors, accelerometers, magnetometers, or proximity sensors to detect a consumption event and/or dispensing event, which can trigger the wireless sensing module 115 to power on and perform the verification techniques described herein (e.g., immediately or in periodic intervals). In such examples, the wireless sensing module 115 can operate in a low power state until a triggering event is detected by the sensor(s) in order to save power. When a triggering event (e.g., a consumption or dispensing event) is detected, the event may either be logged in local memory to enable the wireless sensing module 115 to perform a verification upon powering up, or may trigger the module 115 to power up and monitor and verify the event accordingly.

Based on a consumption event trigger, the wireless sensing module 115 can reference the medication schedule of the user (e.g., based on the current time and date) and analyze reflected wireless signals from the medication organizer 110 to determine whether the user has consumed medication from the correct compartment 112, and consumed the correct medication delivery means from that compartment 112. Thus, each time the user takes medication from the medication organizer 110, the wireless sensing means 115 can be triggered to perform the consumption verification methods described herein.

In further implementations, the wireless sensing module 115 can be triggered to verify the contents of each compartment 112 of the medication organizer 110 when a dispensing event is detected. For example, the wireless sensing module 115 can identify when contents are entering at least one of the compartments 112, which can indicate that the medication dispenser 105 is refilling the medication organizer 110. In various aspects, the medication schedule of the user can indicate the types and amounts of medication delivery means that should be present in each compartment 112 at any given time, or after a dispensing event. Accordingly, using the medication schedule of the user, the wireless sensing module 115 can detect a dispensing event and analyze the reflected wireless signals from each compartment 112 of the medication organizer 110 for the unique sensor signatures or each medication delivery means. The wireless sensing means 115 may then verify whether the correct types and amounts of medication are present in each compartment 112 of the medication organizer 110.

In further examples, given the granularity of wireless signal sensing technology, the wireless sensing module 115 can further authenticate whether the correct user is taking the medication at each consumption event, or whether an unauthorized individual is taking or stealing medication from the device 100. For example, the wireless sensing module 115 can determine the individual characteristics and traits of the user, such as height, shoulder and torso width, head size and shape, arm length, etc. Upon detecting a consumption event, the wireless sensing module 115 can analyze the wireless signals reflected from the user and determine whether the correct user is consuming the medications from the medication organizer 110. If the wireless sensing module 115 determines that an incorrect user is taking the medication, the module 115 can transmit an alert to the computing device 120 to indicate that an unauthorized individual has taken medication delivery means from the medication organizer 110 and/or dispenser 105. In certain examples, the wireless sensing module 115 can further indicate the exact amount and type of medication taken by the unauthorized individual based on the unique sensor signatures of the medication delivery means still in the organizer 110 as compared to those indicated in the user's medication schedule.

In response to each consumption and/or dispensing event, the wireless sensing module 115 can transmit an alert or confirmation to the computing device 120 of the user (e.g., via a health alert app 125 executing thereon). For example, when the sensing module 115 determines that an incorrect amount and/or type of medication is present in a compartment 112—which can indicate a consumption anomaly or a dispensing anomaly—the wireless sensing module 115 can transmit an alert indicating the anomaly. In some aspects, the wireless sensing module 115 can determine the specific details of the anomaly, such as a specific missing medication within a compartment 112, or a medication still present in a compartment 112 that should have been consumed.

In some examples, the wireless sensing module 115 can transmit a confirmation notification to the computing device 120 of the user or an associate of the user (e.g., a caretaker, family member, health care provider, etc.) to indicate that a dispensing event or a consumption event has been verified. For example, the health alert app 125 can execute as a background application on the computing device 120. Upon detection of a consumption or dispensing event, the wireless sensing module 115 can confirm that a correct amount and/or type of medication was consumed or dispensed into the medication organizer 110, and upon confirmation, transmit a confirmation notification to the computing device 120 (e.g., via the health alert app 125). The notification can comprise a push notification, text message, e-mail, etc.

Additionally or alternatively, the wireless sensing module 115 can transmit an alert notification when a dispensing event or consumption event is determined to be inaccurate. For example, upon detection of a consumption or dispensing event, the wireless sensing module 115 can determine that an incorrect amount and/or type of medication was consumed or dispensed into the medication organizer 110, and in response, transmit an alert notification to the computing device 120 (e.g., via the health alert app 125), which can comprise a push notification, text message, e-mail, etc.

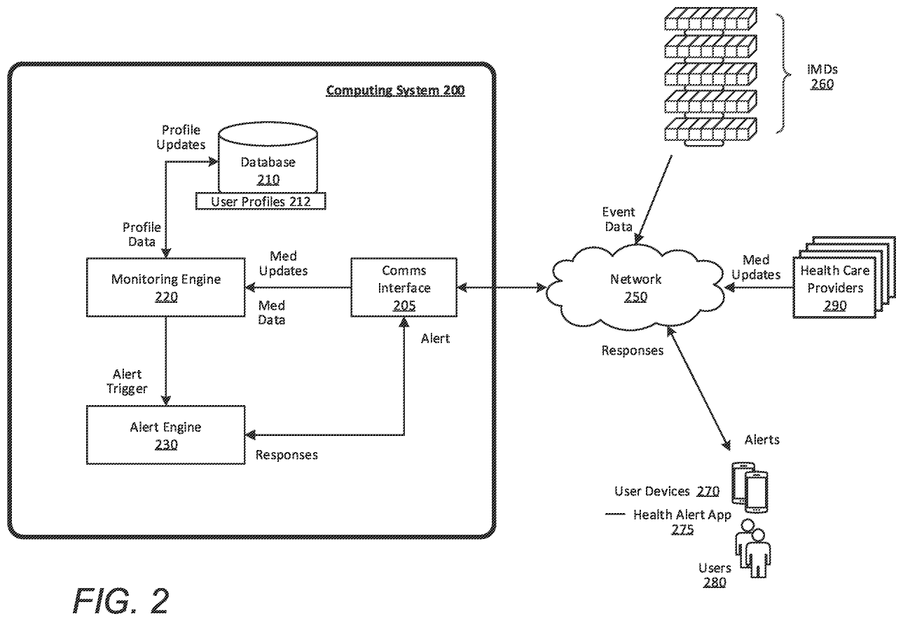

In variations, the wireless sensing module 115 can be triggered to collect wireless signal data when a consumption and/or dispensing event is detected, and transmit the wireless signal data to a remote computing system 130 for analysis. In such examples, the remote computing system 130 can facilitate a remote medication monitoring service for any number of users. Further description of the remote medication monitoring service is described below with respect to FIG. 2.

### System Description

FIG. 2 is a block diagram illustrating a network-based computing system 200 implementing a remote medication monitoring service, according to examples described herein. In various implementations, the computing system 200 can include a database 210 comprising user profiles 212 of the users 280 of the service. Each user profile 212 can indicate the personal details of a corresponding user 280, such as a name, address, age, etc. The user profile 212 may also store a medication schedule for each user 280, which can indicate the amounts and the types of medication to be consumed by the corresponding user 280 at specified times. In certain examples, the computing system 200 can include a communication interface 205 that enables the computing system 200 to communicate, over a network 250, with the computing systems of health care providers 290 in order to, for example, receive the medication schedules of the users 280 and any updates to the medication schedules.

According to examples described herein, the computing system 200 can further communicate, over the network 250, with intelligent medication devices 260, which can correspond to the intelligent medication device 100 described with respect to FIG. 1. The intelligent medication devices 260 can reside with the users 280, and as described above, can detect dispensing and/or consumption events. As provided herein, the wireless signal analysis and verification steps may be performed, in whole or in part, by the wireless sensing modules 115 of the intelligent medication devices 260, a monitoring engine 220 of the computing system 200, or a combination of the intelligent medication devices 260 and the monitoring engine 220. Thus, the event data received from the intelligent medication devices 260 can comprise raw wireless signal data, filtered signal data, a timestamp, an identifier of the user 280 or device 260, and/or notifications corresponding to the confirmations and alerts described above.

In certain implementations, the monitoring engine 220 can receive event data from an intelligent medication device 260 of a user 280, where the event data corresponds to a dispensing or consumption event. The monitoring engine 220 can identify a user 280 from the event data and perform a lookup in the database 210 for the user profile 212 of the user 280. Using the profile data, the monitoring engine 220 can determine a medication schedule of the user 280 and can perform the verification techniques described herein. Specifically, for each dispensing and/or consumption event, the monitoring engine 220 can process the event data to verify the user's identity, verify that the correct amounts and types of medication were consumed from a specified compartment, and/or verify that the correct amounts and types of medication were dispensed into the appropriate compartments of the medication organizer.

Upon detecting an anomaly (e.g., an unauthorized user or an anomalous consumption or dispensing event), the monitoring engine 220 can transmit an alert trigger to an alert engine 230 of the computing system 200. The alert engine 230 can generate specified notifications for each user 280 to indicate the nature and details of each anomaly. For example, the alert engine 230 can specify in an alert that an unauthorized user has taken medication delivery means from the intelligent medication device 260 of the user 280, and transmit the alert to the computing device 270 of the user 280 (e.g., via the health alert app 275). Upon transmitting the alert, the health alert app 275 can initiate an interactive mode that enables the user 280 to communicate with the alert engine 230 to, for example, report a theft or authorize the individual (e.g., indicate that the individual is a family member that is delivering the medication to the user 280).

Additionally or alternatively, upon detecting incorrect consumption or dispensing, the alert engine 230 can transmit an alert that can comprise a simple notification indicating the anomaly. In variations, the alert engine 230 can indicate the specifics of the event, including the specific medication delivery means that have wrongly been consumed or that have not been consumed by the user 280 based on the user's medication schedule. In further variations, the alert engine 230 can indicate the specific medication delivery means that are missing from specified compartments—or those that should not be in specified compartments—of the intelligent medication device 260 of the user 280 (e.g., subsequent to a dispensing event and/or consumption event).

In further implementations, the computing system 200 can communicate with the health care providers 290 of the users 280 to receive any medication updates, such as new medications and/or new dosages of each of the users 280. Upon receiving the medication updates, the monitoring engine 220 can update the medication schedule in the user profiles 212 of the relevant users 280. Thereafter, upon receiving an indication of a consumption and/or dispensing event, the monitoring engine 220 can correlate the event data from the intelligent medication device 260 with the updated medication schedule of the user 280.

It is contemplated that each medication delivery means has a unique set of attributes, which the computing system 200 can represent or associate with a unique sensor signature (e.g., via an initial scan or test of the delivery means). Using the wireless signal processing techniques described herein, the monitoring engine 220 can analyze wireless signal data to identify any specified medication delivery means (e.g., on a millimeter scale). Thus, the users 280 of the remote medication monitoring service can be provided with personalize assistance and security regarding their medication schedules. It is further contemplated that the computing system 200 can communicate medication consumption data to the health care providers 290 of the users 280 to indicate whether the users 280 have been consuming their medications properly.

Methodology

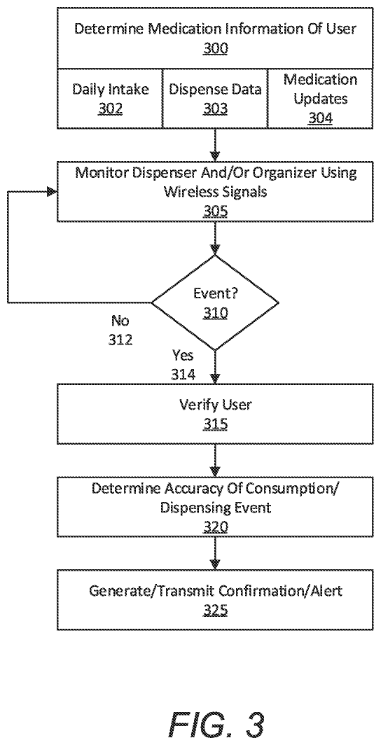

FIG. 3 is a flow chart describing a method of wireless monitoring and verifying medication events and alerting users when anomalies occur, according to various examples. In the below description of FIG. 3, reference may be made to reference characters representing like features as shown and described with respect to FIGS. 1 and 2. Furthermore, the steps described in connection with FIG. 3 may be performed by a wireless sensing module 115 of an intelligent medication device 100, a remote computing system 200, or a combination of a wireless sensing module 115 and a remote computing system 200 (referred to hereafter as the signal processing device).

Referring to FIG. 3, the signal processing device can determine medication information of a user 280 (300). The medication information can correspond to a medication schedule of the user 280, and can indicate a daily intake of the user 280 (302), dispensing information indicating when and an amount and type of medication to be dispensed into each compartment 112 of the user's medication organizer 110 (303), and any medication updates, which can be received from the user 280 or a health care provider 290 of the user 280 (304). The signal processing device can monitor the dispenser 105 and/or organizer 110 of the user's intelligent medication device 100 using wireless signals (305). As described herein, the wireless signals may be generated by a signal generator in the wireless sensing module 115 of the intelligent medication device 100, or can comprise reflected signals from any wireless device (e.g., Wi-Fi signals from a router, smartphone, tablet computer, smart home device, etc.).

At any given time, the signal processing device can determine whether an event has occurred (310). As described above, the event can comprise a consumption event or a dispensing event. If an event has not occurred (312), the signal processing device may continue to monitor either actively or in a background state. If a consumption event has been detected (314), the signal processing device can analyze the wireless signals reflected from the individual to verify whether the correct user 280 is taking the medication (315). If a consumption or dispensing event has been detected (314), the signal processing engine can process the wireless signals reflecting from the medication delivery means within each compartment 112 of the intelligent medication device 100 to determine an accuracy of the consumption event and/or dispensing event (320). In further implementations, the signal processing device can verify whether the user 280 has opened the correct compartment 112 of the medication organizer 110. Based on the analysis, the signal processing device can generate and transmit a confirmation that the event was accurate, or an alert indicating an anomaly in the event (325).

User Computing Device

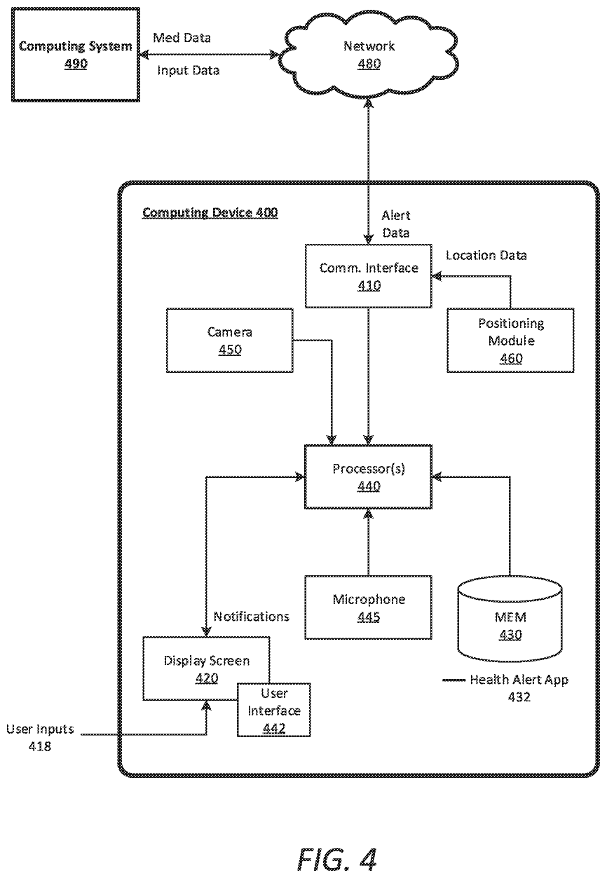

FIG. 4 illustrates a computing device for communicating with an intelligent medication device and/or computing system, according to examples described herein. In many implementations, the computing device 400 can comprise a mobile computing device, such as a smartphone, tablet computer, laptop computer, personal computer, VR or AR headset device, and the like. In certain examples, the computing device 400 can include telephony features such as a microphone 445, a camera 450, and a communication interface 410 to communicate with the computing system 490 using any number of wireless communication protocols. The computing device 400 can further include a positioning module 460 (e.g., GPS) and/or an inertial measurement unit that includes one or more accelerometers, gyroscopes, or magnetometers.

In certain aspects, the computing device 400 can store a designated health alert application 432 in a local memory 430. The computing device 400 can further access one or more networks 480 via a web browser. In variations, the memory 430 can store additional applications executable by one or more processors 440 of the computing device 400, enabling access and interaction with one or more servers over the one or more networks 480.

Additionally, the computing device 400 can be operated by a user 280 through execution of the health alert application 432. In various examples, the user 280 can select the health alert application 432 via a user input 418 on the display screen 420, which can cause the application 432 to be executed by the processor 440. In response, an interactive user interface 442 can be generated on the display screen 420, which can display details of alerts, notifications, and personal medication information of the user 280.

As provided herein, the application 432 can enable a communication link over one or more networks 480 with the computing system 490, such as the computing system 200 as shown and described with respect to FIG. 2. The processor 440 can generate user interface features 442 using content data received from the computing system 490 over network 480. Furthermore, as discussed herein, the application 432 can enable the computing system 490 to cause the user interface 422 to be displayed on the display screen 420.

The computing device 400 can communicate over the network 480 to provide the computing system 490 with, for example, medication data, medication updates, and input data representing user inputs via the user interface 442. The computing device 400 can further communicate with the computing system 490 to receive notifications based on a consumption and/or dispensing anomaly or an unauthorized access alert indicating potential theft of medication.

Hardware Diagram

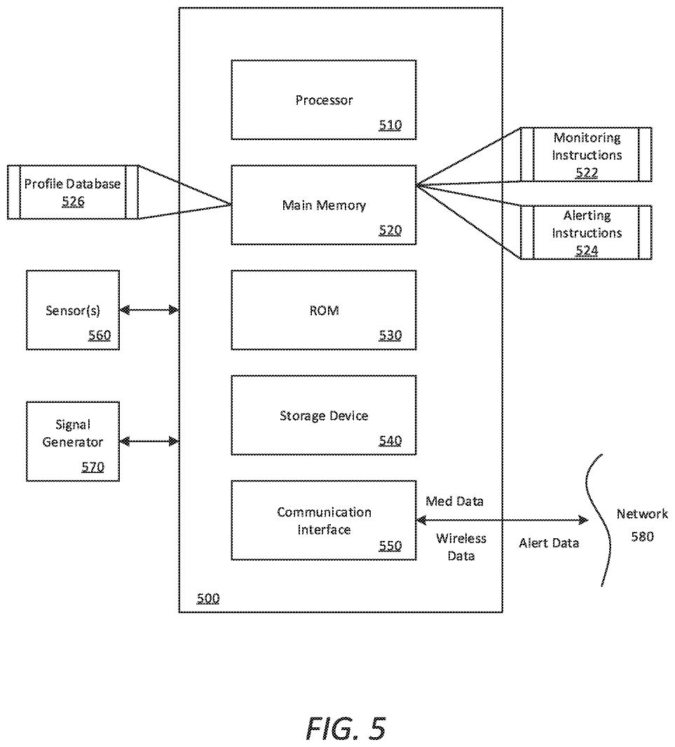

FIG. 5 is a block diagram that illustrates a computer system 500 upon which examples described herein may be implemented. A computer system 500 can be implemented on, for example, a server or combination of servers. For example, the computer system 500 may be implemented as part of a network-based service, such as described in FIGS. 1 through 4. In the context of FIGS. 1 and 2, the wireless sensing module 115 and/or the computing system 200 may be implemented using a computer system 500 such as described by FIG. 5. The wireless sensing module 115 of FIG. 1 and the computing system 200 of FIG. 2 may also be implemented using a combination of multiple computer systems 500 as described in connection with FIG. 5.

In one implementation, the computer system 500 includes processing resources 510, a main memory 520, a read-only memory (ROM) 530, a storage device 540, and a communication interface 550. The computer system 500 includes at least one processor 510 for processing information stored in the main memory 520, such as provided by a random-access memory (RAM) or other dynamic storage device, for storing information and instructions which are executable by the processor 510. The main memory 520 also may be used for storing temporary variables or other intermediate information during execution of instructions to be executed by the processor 510. The computer system 500 may also include the ROM 530 or other static storage device for storing static information and instructions for the processor 510. A storage device 540, such as a magnetic disk or optical disk, is provided for storing information and instructions.

The communication interface 550 enables the computer system 500 to communicate with one or more networks 580 (e.g., cellular network) through use of the network link (wireless or wired). Using the network link, the computer system 500 can communicate with one or more computing devices, one or more servers, and/or one or more databases. In various examples, the computer system 500 can further include or communicate with one or more wireless signal sensors 560 and/or one or more wireless signal generators 570 (e.g., a Wi-Fi signal generator) to perform the operations described throughout the present disclosure. In accordance with examples provided herein, the memory 520 can store a profile database 526 comprising user profiles. In various examples, the executable instructions stored in the memory 520 can include monitoring instructions 522 alerting instructions 524.

By way of example, the instructions and data stored in the memory 520 can be executed by the processor 510 to implement the functions of an example computing system 200 of FIG. 2 and/or the wireless sensing module 115 of FIG. 1. In various examples, the processor 510 can execute the monitoring instructions 522 to process wireless signal data to detect events, verify whether an authorized user has opened a medication organizer, and determine whether a correct amount and type of medication was consumed or dispensed into the appropriate compartments of the medication organizer. The processors 510 can further execute the alerting instructions 524 to generate and transmit confirmations and/or alerts upon detecting consumption and/or dispensing event, as described above.

Examples described herein are related to the use of the computer system 500 for implementing the techniques described herein. According to one example, those techniques are performed by the computer system 500 in response to the processor 510 executing one or more sequences of one or more instructions contained in the main memory 520. Such instructions may be read into the main memory 520 from another machine-readable medium, such as the storage device 540. Execution of the sequences of instructions contained in the main memory 520 causes the processor 510 to perform the process steps described herein. In alternative implementations, hard-wired circuitry may be used in place of or in combination with software instructions to implement examples described herein. Thus, the examples described are not limited to any specific combination of hardware circuitry and software.

It is contemplated for examples described herein to extend to individual elements and concepts described herein, independently of other concepts, ideas or systems, as well as for examples to include combinations of elements recited anywhere in this application. Although examples are described in detail herein with reference to the accompanying drawings, it is to be understood that the concepts are not limited to those precise examples. As such, many modifications and variations will be apparent to practitioners skilled in this art. Accordingly, it is intended that the scope of the concepts be defined by the following claims and their equivalents. Furthermore, it is contemplated that a particular feature described either individually or as part of an example can be combined with other individually described features, or parts of other examples, even if the other features and examples make no mentioned of the particular feature. Thus, the absence of describing combinations should not preclude claiming rights to such combinations.

What is claimed is:

1. An intelligent medication device comprising:
a medication organizer comprising a plurality of medication compartments, each of the plurality of medication compartments to hold a corresponding set of medication delivery means at any given time; and
a wireless sensing module within wireless range of the medication organizer, the wireless sensing module comprising:
one or more wireless signal sensors;
a network communication interface to communicate, over one or more networks, with one or more computing devices;
one or more processors; and
a memory storing instructions that, when executed by one or more processors, cause the wireless sensing module to:
determine a medication schedule of a user of the medication organizer;
monitor, using the one or more wireless signal sensors, the corresponding set of medication delivery means in each of the plurality of compartments for medication consumption events by the user, wherein the monitoring comprises analyzing reflected wireless signals from each of the plurality of compartments; and
based on the medication schedule of the user, verify each of the medication consumption events to determine whether an anomaly has occurred.

2. The intelligent medication device of claim 1, wherein the executed instructions further cause the wireless sensing module to:
in response to determining that an anomaly has occurred, transmit, over the one or more networks, an alert indicating the anomaly to the computing device of the user.

3. The intelligent medication device of claim 1, wherein the medication delivery means comprise at least one of pills, liquid capsules, or medication delivery devices.

4. The intelligent medication device of claim 1, further comprising:
a wireless signal generator;
wherein the executed instructions further cause the wireless sensing module to:
generate, using the wireless signal generator, a wireless signal that penetrates each compartment of the plurality of compartments of the medication organizer;
wherein the reflected wireless signals from each of the plurality of compartments correspond to the wireless signal generated by the wireless signal generator.

5. The intelligent medication device of claim 1, further comprising:
a dispenser;
wherein the executed instructions further cause the wireless sensing module to:
monitor, using the one or more wireless signal sensors, the plurality of compartments of the medication organizer for dispensing events, each dispensing event comprising the corresponding set of medication delivery means for each compartment being dispensed into the medication organizer by the dispenser.

6. The intelligent medication device of claim 5, wherein the executed instructions further cause the wireless sensing module to:
verify, using the medication schedule of the user, an accuracy of each detected dispensing event.

7. The intelligent medication device of claim 6, wherein the executed instructions further cause the wireless sensing module to:
when a particular dispensing event comprises an inaccuracy, transmit, over the one or more networks, an alert to the computing device of the user to indicate the inaccuracy.

8. The intelligent medication device of claim 1, wherein each medication delivery means in the corresponding set of medication delivery means in each of the plurality of compartments corresponds to a sensor signature, and wherein the executed instructions cause the wireless sensing module to verify each of the medication consumption events by comparing the sensor signatures of the medication delivery means in each of the plurality of compartments with the medication schedule of the user.

9. A computing system implementing a remote medication monitoring and verification service, the computing system comprising:
a database storing user profiles of users of the remote medication monitoring and verification service, each user profile indicating a medication schedule of a respective user of the remote medication monitoring and verification service;
a network communication interface to communicate, over one or more networks, with (i) wireless sensing modules of intelligent medication devices of the users of the remote medication monitoring and verification service, and (ii) computing devices of the users;
one or more processors; and
a memory storing instructions that, when executed by the one or more processors, cause the computing system to:
receive, over the one or more networks, event data indicating a medication event sensed by the wireless sensing module of the respective user, the event data indicating one of (i) a medication consumption event by the respective user, or (ii) a medication dispensing event corresponding to medication being dispensed into a medication organizer of the respective user;
analyze the event data using the medication schedule of the respective user to verify the medication event; and
when an error has occurred associated with the medication event, transmit, over the one or more networks, an alert to the computing device associated with the respective user, the alert indicating the error.

10. The computing system of claim 9, wherein the event data comprises raw sensor data from the wireless sensing module of the respective user.

11. The computing system of claim 10, wherein the executed instructions further cause the computing system to:
based on the medication schedule of the respective user, analyze the raw sensor data to determine whether each particular compartment of the medication organizer of the respective user has an accurate set of medication delivery means;
wherein the error corresponds to an inaccurate set of medication delivery means in the particular compartment.

12. The computing system of claim 11, wherein each medication delivery means in a corresponding set of medication delivery means of each compartment of the medication organizer corresponds to a sensor signature, and wherein the executed instructions cause the computing system to verify the medication event by comparing the sensor signatures of each medication delivery means in each corresponding set of medication delivery means of the medication organizer with the medication schedule of the user.

13. The computing system of claim 9, wherein the network communication interface further communicates, over the one or more networks, with one or more health care providers of the respective user, and wherein the executed instructions cause the computing system to determine the medication schedule of the respective user based on communications with the one or more health care providers.

14. The computing system of claim 13, wherein the executed instructions further cause the computing system to:
receive, over the one or more networks, a medication update from the one or more health care providers of the respective user; and
update the medication schedule in the user profile of the respective user to include the medication update.

15. The computing system of claim 9, wherein the user profile of the respective user further indicates an emergency contact, and wherein the executed instructions cause the computing system to transmit the alert to the computing device of the emergency contact when the error has occurred.

16. A computer implemented method of monitoring medication events, the method being performed by one or more processors of a signal processing device and comprising:
determining a medication schedule of a user of a medication organizer;
monitoring, using one or more wireless signal sensors, a corresponding set of medication delivery means in each of a plurality of compartments of the medication organizer for medication consumption events by the user, wherein the monitoring comprises analyzing reflected wireless signals from each of the plurality of compartments; and
based on the medication schedule of the user, verify each of the medication consumption events to determine whether an anomaly has occurred.

17. The method of claim 16, further comprising:
in response to determining that an anomaly has occurred, transmit, over the one or more networks, an alert indicating the anomaly to the computing device of the user.

18. The method of claim 16, wherein the medication delivery means comprise at least one of pills, liquid capsules, or medication delivery devices.

19. The method of claim 16, further comprising:
generating, using a wireless signal generator, a wireless signal that penetrates each compartment of the plurality of compartments of the medication organizer;
wherein the reflected wireless signals from each of the plurality of compartments correspond to the wireless signal generated by the wireless signal generator.

20. The method of claim 16, further comprising:
monitoring, using the one or more wireless signal sensors, the plurality of compartments of the medication organizer for dispensing events, each dispensing event comprising the corresponding set of medication delivery means for each compartment being dispensed into the medication organizer by the dispenser.
